(19) United States
(12) Patent Application Publication
(10) Pub. No.: US 2024/0382716 A1
(43) Pub. Date: Nov. 21, 2024

(54) MODULE FOR TRANSLATING A PLURALITY OF ELONGATE FLEXIBLE MEDICAL INSTRUMENTS, COMPRISING A DEVICE FOR CONVERGENCE TOWARDS A COMMON TRACK

(71) Applicant: ROBOCATH, ROUEN (FR)

(72) Inventor: Alexis BELOUIN, POUILLÉ LES COTEAUX (FR)

(21) Appl. No.: 18/692,177

(22) PCT Filed: Sep. 15, 2022
(86) PCT No.: PCT/EP2022/075658

§ 371 Date: Mar. 14, 2024

(30) Foreign Application Priority Data
Sep. 17, 2021 (FR) 2109769

(51) Int. Cl. A61M 25/01

(52) U.S. Cl. CPC A61M 25/0116

(57) ABSTRACT

The invention relates to a drive module (3) of a catheter robot, for driving the translation of a plurality of elongate flexible medical instruments, comprising: a mechanism for driving the translation of a plurality of elongate flexible medical instruments; and a device (5) for the convergence, between a plurality of guide tracks (32) for respectively guiding the translation of a plurality of elongate flexible medical instruments, towards a common exit track (33) shared by these elongate flexible medical instruments, comprising: track connectors which are respectively located between these guide tracks (32) and the exit track (33) and which ensure continuity in the guidance of these elongate flexible medical instruments respectively between these guide tracks (32) and the exit track (33), these track connectors being retractable.

### FIELD OF THE INVENTION

The invention relates to the technical field of drive modules of a catheter robot, for driving the translation of a plurality of elongate flexible medical instruments, comprising a device for the convergence, between a plurality of tracks for respectively guiding the translation of a plurality of elongate flexible medical instruments, towards a common exit track shared by these elongate flexible medical instruments.

### TECHNOLOGICAL BACKGROUND OF THE INVENTION

From prior art in the patent application filed under number FR2101185 on Feb. 8, 2021, it is known to have a convergence device which comprises track connectors between a plurality of respective tracks for guiding the translation of a plurality of elongate flexible medical instruments towards a common exit track shared by these elongate flexible medical instruments. The entire convergence device is fixed relative to the base of the drive module.

From time to time, the practitioner needs to create a “fixed point” which may be in order to move one elongate flexible medical instrument while keeping another elongate flexible medical instrument fixed, for example either moving the catheter while keeping the catheter guide fixed, or for example moving the catheter guide while keeping the catheter fixed.

This “fixed point” is usually created by the practitioner by pressing a finger on one of the elongate flexible medical instruments, the one to be kept in place.

This “fixed point” is useful in particular in the following situations:
when installing the catheter guide in the drive module,
when loading the catheter onto its catheter guide,
when unloading the catheter from its catheter guide.

A disadvantage of this prior art lies in the fact that, when the practitioner needs to create a “fixed point”, he or she is hindered by the respective track connectors which connect a plurality of respective tracks for guiding the translation of a plurality of elongate flexible medical instruments towards a common exit track shared by these elongate flexible medical instruments. However, eliminating these connections could lead to poorer translational guidance of these elongate flexible medical instruments, or even to creating a loop in all or part of these elongate flexible medical instruments (i.e. instead of translational movement along its translation path, the elongate flexible medical instrument loops or travels off course before correctly resuming its translation path.

### OBJECTS OF THE INVENTION

The aim of the present invention is to provide a drive module of a catheter robot, for driving the translation of a plurality of elongate flexible medical instruments, which at least partially overcomes the above disadvantages.

More particularly, the invention aims to provide a drive module of a catheter robot, for driving the translation of a plurality of elongate flexible medical instruments, which on the one hand continues to guide the translation of these elongate flexible medical instruments properly, while on the other hand part facilitates the work of the practitioner by making it easier to create a “fixed point”.

The invention proposes making the track connectors retractable. The track connectors are located between these guide tracks and the exit track, respectively, and ensure continuity in the guidance of these elongate flexible medical instruments respectively between their guide tracks and the exit track, while the practitioner is creating a “fixed point”. Advantageously, only the track connectors are retractable, the guide tracks not being retractable. Advantageously, a part of the exit track is also made retractable, but only a part.

To this end, the invention proposes a drive module of a catheter robot, for driving the translation of a plurality of elongate flexible medical instruments, comprising: a mechanism for driving the translation of a plurality of elongate flexible medical instruments; a convergence device for the convergence, between a plurality of tracks for respectively guiding the translation of a plurality of elongate flexible medical instruments, towards a common exit track shared by these elongate flexible medical instruments, comprising: track connectors which are respectively located between these guide tracks and the exit track and which ensure continuity in the guidance of these elongate flexible medical instruments respectively between these guide tracks and the exit track;
characterized in that: these track connectors are retractable, such that, in the non-retracted position, the track connectors respectively form channels for guiding the translation of these elongate flexible medical instruments, and in the retracted position, the track connectors no longer respectively form channels for guiding the translation of the elongate flexible medical instruments.

In the retracted position, the respective track connectors no longer form channels for guiding the translation of these elongate flexible medical instruments, but leave open an access to these elongate flexible medical instruments by a practitioner's finger, allowing said finger easily to pinch one or more of these elongate flexible medical instruments.

In the non-retracted position, also called the deployed position, the track connectors respectively form channels for guiding the translation of these elongate flexible medical instruments, thus protecting the elongate flexible medical instruments by limiting or preventing access to the elongate flexible medical instruments.

According to preferred embodiments, the invention comprises one or more of the following features which may be used separately or in combinations of some of them or in a combination of all of them.

Preferably, these track connectors are: integrally secured together so as to be retractable simultaneously in a single operation, or are all integrated into a same part which itself is retractable.

Retraction of the track connectors is thus made even simpler and even easier, which makes it even easier for the practitioner to create the “fixed point”.

This operation may in particular be either a remote control of a suitable actuator, or a direct mechanical action by a practitioner's finger.

Preferably, these track connectors are retractable towards the inside of the drive module, or towards the bottom of the drive module when the drive module is in position and in its orientation for use.

Retraction of the track connectors is thus made even simpler and even easier, which makes it even easier for the practitioner to create the “fixed point”.

In addition, there is less risk of damage to the retracted parts during creation of the “fixed point” by the practitioner.

Preferably, these track connectors are retractable by withdrawing into at least one orifice of the drive module, while remaining guided by one or more walls of this orifice during their withdrawal.

Retraction of the track connectors is thus made even simpler and even more robust, with the entire retraction system less likely to wear out or malfunction over time and during successive uses.

Preferably, these track connectors are retractable by withdrawing into at least two orifices of the drive module which are separated by a fixed platform that does not withdraw into the drive module, while remaining guided by one or more walls of this orifice during their withdrawal as well as by one or more walls of this fixed platform.

Retraction of the track connectors is thus made even simpler and even easier, which makes it even easier for the practitioner to create the “fixed point”, while allowing the track connectors to be retracted all the way to the common exit track or possibly including part of this common exit track.

Preferably, these track connectors are mechanically retractable by a simple pressure exerted on them by the practitioner's finger.

Retraction of the track connectors is thus made even simpler and even easier, which makes it even easier for the practitioner to create the “fixed point”.

Preferably, the convergence device comprises a return element for the purpose of returning or maintaining these track connectors in the non-retractable position, in the absence of pressure being exerted on them.

The default rest position is thus the non-retracted position, the one required most of the time; retraction of the track connectors is only useful from time to time, for example during creation of a “fixed point” by the practitioner.

This return element is for example a spring.

Preferably, these track connectors respectively curve from the plurality of guide tracks towards the common exit track.

Preferably, these track connectors are all integrated as one part in a single contact pad which can be retracted simply by pressing on it.

Retraction of the track connectors is thus made even simpler and even more robust, the entire retraction system being less likely to be degraded or damaged and then to malfunction over time and during successive uses.

Preferably, said contact pad is a single piece of molded plastic comprising: at least one alternation of recesses and protuberances, a plurality of elongate flexible medical instruments being respectively guided in the recesses between the walls formed by the protuberances.

Retraction of the track connectors is thus made even simpler and even more robust, the entire retraction system being less likely to be degraded or damaged and then to malfunction over time and during successive uses.

Preferably, the track connectors form at least one alternation of recesses and protuberances, a plurality of elongate flexible medical instruments being respectively guided in the recesses between the walls formed by the protuberances.

Preferably, the track connectors form at least one alternation of recesses and protuberances, a plurality of elongate flexible medical instruments being respectively guided in the recesses between the walls formed by the protuberances, such that, in the non-retracted position, the protuberances at their periphery are flush with the rest of the drive module.

The track connectors thus do not protrude from the rest of the (external) surface of the drive module, which helps to avoid collisions and the risk of damage to equipment during the practitioner's various manipulations of the elongate flexible medical instruments.

Preferably, the track connectors of the convergence device are grooves made in a surface having the same concave curvature.

Retraction of the track connectors is thus made even simpler and even more robust, the entire retraction system being less likely to be degraded or damaged and then to malfunction over time and during successive uses.

This surface having the same concave curvature has a width adapted to receive a practitioner's finger, thus allowing him or her to easily pinch one or more elongate flexible medical instruments.

The track connectors of the convergence device may also be track connectors formed by protruding walls extending from a given surface.

Preferably, a part of the exit track is also retractable, but only a part.

Having a part of the exit track be retractable makes the creation of a “fixed point” by the practitioner more practical because he or she then has more space. Having only a part be retractable makes it possible to maintain precise and robust guidance over time for the entry into the Y connector, this precise guidance being of particular interest at this location which is a point of convergence for a plurality, and sometimes numerous, elongate flexible medical instruments.

Preferably, the guide tracks are not retractable.

The elongate flexible medical instruments thus remain properly held in place, even when the practitioner is creating a “fixed point”.

Preferably, the drive module comprises: a consumable interface, which includes the convergence device comprising the retractable track connectors, but which does not include the mechanism for driving the translation of a plurality of elongate flexible medical instruments.

Preferably, the guide tracks advantageously successively comprise: a first guide track in which a first catheter is moved; a second guide track in which a first catheter guide is moved, intended to be inserted into the first catheter in the common exit track; a third guide track in which a second catheter guide is moved, intended to be inserted into a second catheter in the common exit track; a fourth guide track in which a second catheter is moved; the first catheter and the second catheter being intended to be next to each other in the common exit track.

Preferably, the mechanism for driving the translation of a plurality of elongate flexible medical instruments is also a mechanism for driving the rotation of least some of these elongate flexible medical instruments.

Preferably, the drive module is movable relative to the base of the catheter robot, and carries a Y connector which the common exit track leads into.

The creation of a “fixed point” is more common for a drive module that is movable relative to the base of the catheter robot than for a drive module that is fixed relative to the base of the catheter robot.

The drive module may also be fixed relative to the base of the catheter robot, and possibly may carry a guide catheter or a micro-catheter.

Other features and advantages of the invention will become apparent upon reading the following description of a preferred embodiment of the invention, given by way of example and with reference to the appended drawings.

### BRIEF DESCRIPTION OF DRAWINGS

FIG. 1 schematically represents an example of a catheter robot according to one embodiment of the invention.

FIG. 2 schematically represents a detailed top view of the distal end of the movable drive module of the catheter robot of FIG. 1.

FIG. 3 schematically represents the convergence device in the non-retracted position according to one embodiment of the invention.

FIG. 4 schematically represents the convergence device in the retracted position according to one embodiment of the invention.

FIG. 5 schematically represents an isolated view of a contact pad of the convergence device according to one embodiment of the invention.

### DETAILED DESCRIPTION OF EMBODIMENTS OF THE INVENTION

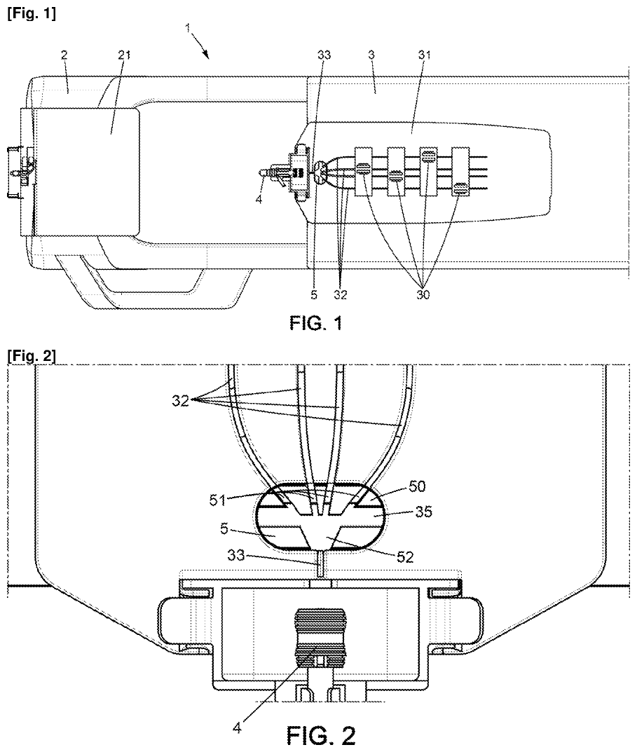

As illustrated in FIG. 1, a catheter robot 1 comprises a fixed drive module 2 and a movable drive module 3 which is movable in translation relative to fixed module 2. Fixed drive module 2 and movable drive module 3 each comprise a mechanism for driving the translation and rotation of an elongate flexible medical instrument. This mechanism for driving a medical instrument may for example be formed by robotic fingers, as described in patent FR3022147, or the patent application filed under number FR2014222 on Dec. 29, 2020.

Fixed drive module 2 comprises a consumable interface 21 in the form of a plate or cassette which is intended to receive a guide catheter, a micro-catheter, or a catheter carrying a balloon or a stent. Movable drive module 3 also comprises a consumable interface 31 in the form of a plate or cassette which is intended to receive a catheter guide and a catheter which carries a balloon or a stent. Consumable interfaces 21 and 31 may in particular comprise consumable contact pads 30 which are intended to form a sterile interface between the fingers of fixed and movable drive modules 2 and 3 and the elongate flexible medical instruments, as described in patent FR3022147. For simplification, contact pads 30 are only shown for movable drive module 3.

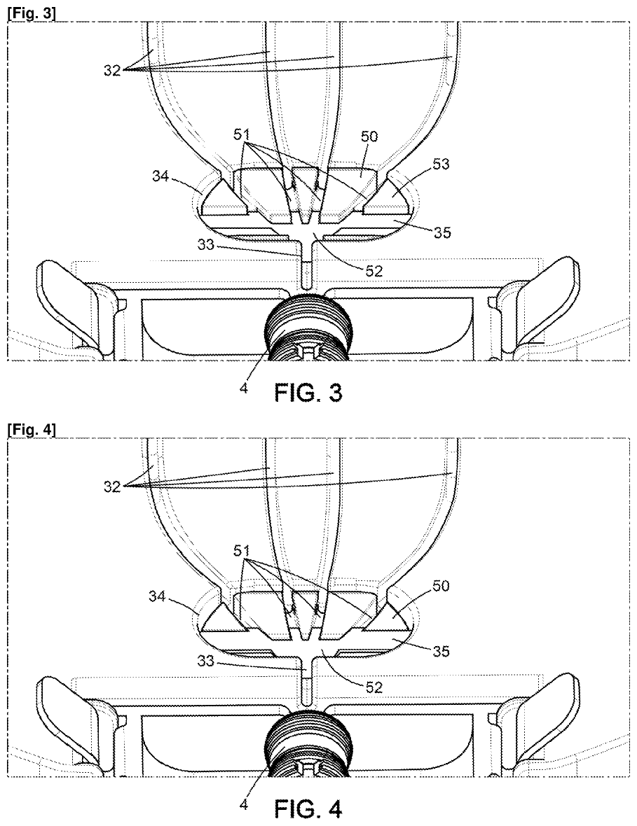

As can be seen in FIGS. 1 to 4, guide tracks 32 are formed on consumable interface 31 in order to receive and guide the elongate flexible medical instruments installed on consumable interface 31. In the variant illustrated in FIGS. 1 to 4, guide tracks 32 are formed by grooves made in consumable interface 31. However, according to another possible variant, guide tracks 32 may be formed by walls protruding from consumable interface 31. In the illustrated embodiment, four guide tracks 32 are formed on consumable interface 31; the number of guide tracks 32 may vary, however.

As can be seen in FIG. 1, a Y connector 4 is fixed on the distal end of consumable interface 31. The distal end corresponds to the end furthest from the practitioner and closest to the patient, while the proximal end corresponds to the end closest to the practitioner and farthest from the patient. The four guide tracks 32 are parallel on a proximal and central part of consumable interface 31, then converge on a distal part of consumable interface 31 towards Y connector 4 in order to form a single common exit track 33 leading into Y connector 4.

In order to ensure proper guidance of medical instruments, from their respective guide tracks 32 towards Y connector 4, consumable interface 31 comprises a convergence device 5 located between guide tracks 32 and Y connector 4. Convergence device 5 forms track connectors which connect guide tracks 32 to exit track 33, thus ensuring continuity in the guidance by guide tracks 32 and exit track 33.

Consumable interface 31 comprises four guide tracks 32; said consumable interface 31 is configured for manipulating four medical instruments, for example two guides and two catheters carrying a balloon or a stent. To enable the practitioner to perform “fixed point” manipulation, in particular in order to install a catheter on a guide, convergence device 5 is retractable. Convergence device 5 is thus movable between a deployed position in which convergence device 5 is extending guide tracks 32, and a retracted position in which convergence device 5 leaves room for the practitioner's finger.

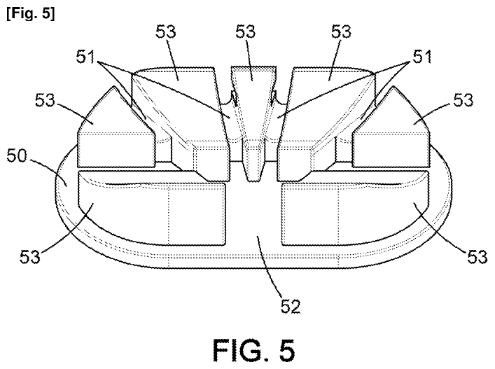

As can be seen in particular in FIGS. 2 to 5, convergence device 5 is formed of a contact pad 50 in which four proximal guide grooves 51 are formed, these proximal guide grooves 51 being intended to extend guide tracks 32. In order to move convergence device 5 from its deployed position to its retractable position, contact pad 50 withdraws into consumable interface 31 when the practitioner presses on it with a finger. A return element, for example a spring, is installed inside consumable interface 31 in order to cause contact pad 50 to come back out when the practitioner stops pressing on contact pad 50, thus causing convergence device 5 to move from its retracted position to its deployed position. Contact pad 50 also comprises a distal guide groove 52 which forms a portion of exit track 33.

Contact pad 50 is installed in an orifice 34 formed in consumable interface 31, at the point of convergence of guide tracks 32. Orifice 34 is separated into two parts by a platform 35 forming a plane at the same level as the bottom of guide tracks 32. This platform is located in a central part of orifice 34, and is intended to form a flat surface adapted to allow the practitioner's finger to pinch the medical instruments against platform 35. The platform comprises a shape complementary to the shape of contact pad 50. Platform 35 remains fixed while contact pad 50 is retracting or returning outwards.

Walls 53 which form the various proximal guide grooves 51 and distal guide groove 52 hat a top which curves inward towards the center of contact pad 50, such that the face of walls 53 which is located facing the contours of orifice 34 is flush with the upper external surface of consumable interface 31 when contact pad 50 is in the deployed position, and the face of walls 53 which is oriented towards the center of contact pad 50 is flush with platform 35 when contact pad 50 is in the retracted position. The shape thus formed by contact pad 50 due to the curved shape of the top of walls 53 is adapted to receive the complementary curved shape of the practitioner's finger.

In one possible variant of catheter robot 1, fixed drive module 2 and consumable interface 21 are configured to drive a single elongate flexible medical instrument, for example a guide catheter. In this variant, the presence of a convergence device 5 on consumable interface 21 is unnecessary. However, in another variant in which fixed drive module 2 and consumable interface 21 are configured to drive a plurality of elongate flexible medical instruments, consumable interface 21 may comprise a convergence device 5 which is similar to convergence device 5 installed on consumable interface 31.

Furthermore, according to one possible variant, catheter robot 1 comprises a plurality of movable drive modules 3 which are placed one after the other.

According to one possible variant, the transition between the deployed and retracted position of convergence device 5 is controlled by an actuator which is controlled by a human-machine interface such as a remote control or any other type of buttons.

Of course, the invention is not limited to the examples and the embodiment described and represented, but is capable of numerous variants accessible to those skilled in the art.

1. Drive module of a catheter robot, for driving the translation of a plurality of elongate flexible medical instruments, comprising:
a mechanism for driving the translation of a plurality of elongate flexible medical instruments,
a convergence device for the convergence, between a plurality of guide tracks for respectively guiding the translation of a plurality of elongate flexible medical instruments, towards a common exit track shared by these elongate flexible medical instruments, comprising:
track connectors which are respectively located between these guide tracks and the exit track and which ensure continuity in the guidance of these elongate flexible medical instruments respectively between these guide tracks and the exit track,
characterized in that:
these track connectors are retractable, such that:
in the non-retracted position, the track connectors respectively form channels for guiding the translation of these elongate flexible medical instruments,
in the retracted position, the track connectors no longer respectively form channels for guiding the translation of these elongate flexible medical instruments.

2. Drive module according to claim 1, wherein:
these track connectors are:
integrally secured together so as to be retractable simultaneously in a single operation,
or are all integrated into a same part which itself is retractable.

3. Drive module according to claim 1, wherein:
these track connectors are retractable:
towards the inside of the drive module,
or towards the bottom of the drive module, when the drive module is in position and in its orientation for use.

4. Drive module according to claim 1, wherein these track connectors are retractable by withdrawing into at least one orifice of the drive module, while remaining guided by one or more walls of this orifice during their withdrawal.

5. Drive module according to claim 4, wherein these track connectors are retractable by withdrawing into at least two orifices of the drive module which are separated by a fixed platform that does not withdraw into the drive module, while remaining guided by one or more walls of this orifice during their withdrawal as well as by one or more walls of this fixed platform.

6. Drive module according to claim 4, wherein these track connectors are mechanically retractable by a simple pressure exerted on them by the practitioner's finger.

7. Drive module according to claim 6, wherein the convergence device comprises a return element for the purpose of returning or maintaining these track connectors in the non-retractable position, in the absence of pressure being exerted on them.

8. Drive module according to claim 1, wherein these track connectors respectively curve from the plurality of guide tracks towards the common exit track.

9. Drive module according to claim 1, wherein these track connectors are all integrated as one part in a single contact pad which can be retracted simply by pressing on it.

10. Drive module according to claim 9, wherein:
said contact pad is a single piece of molded plastic, comprising:
at least one alternation of recesses and protuberances, a plurality of elongate flexible medical instruments being respectively guided in the recesses between the walls formed by the protuberances.

11. Drive module according to claim 1, wherein the track connectors form at least one alternation of recesses and protuberances, a plurality of elongate flexible medical instruments being respectively guided in the recesses between the walls formed by the protuberances.

12. Drive module according to claim 11, wherein the track connectors form at least one alternation of recesses and protuberances, a plurality of elongate flexible medical instruments being respectively guided in the recesses between the walls formed by the protuberances, such that, in the non-retracted position, the protuberances at their periphery are flush with the rest of the drive module.

13. Drive module according to claim 1, wherein the track connectors of the convergence device are grooves made in a surface having the same concave curvature.

14. Drive module according to claim 1, wherein the guide tracks are not retractable.

15. Drive module according to claim 1, wherein the drive module comprises:
a consumable interface:
which includes the convergence device comprising the retractable track connectors,
but which does not include the mechanism for driving the translation of a plurality of elongate flexible medical instruments.

16. Drive module according to claim 2, wherein:
these track connectors are retractable:
towards the inside of the drive module,
or towards the bottom of the drive module, when the drive module is in position and in its orientation for use.
